(19) United States
(12) Patent Application Publication
(10) Pub. No.: US 2018/0177432 A1
(43) Pub. Date: Jun. 28, 2018

(54) APPARATUS AND METHOD FOR DETECTION OF BREATHING ABNORMALITIES

(71) Applicant: STRADOS LABS LLC, Philadelphia, PA (US)

(72) Inventors: Yu Kan AU, Philadelphia, PA (US); Tanziyah MUQEEM, Philadelphia, PA (US); Nicholas Shane DELMONICO, Philadelphia, PA (US)

(21) Appl. No.: 15/851,111

(22) Filed: Dec. 21, 2017

Related U.S. Application Data
(60) Provisional application No. 62/439,254, filed on Dec. 27, 2016.

(51) Int. Cl. A61B 5/08; A61B 5/00; A61B 7/04

(52) U.S. Cl. CPC A61B 5/0803; A61B 5/7257; A61M 2016/0036; A61B 7/04; A61B 5/082; A61B 5/0816

(57) ABSTRACT

An apparatus and method are for evaluating respiration. A microphone is placed in contact with a patient's skin and audio is acquired through the microphone. The acquired audio is sampled, processed and stored. At least one sound associated with respiration is identified. Abnormal respiration is identified based on frequency or duration of at least the identified sound.

This application claims priority to U.S. Provisional Application 62/439,254, which is hereby incorporated by reference in its entirety.

### FIELD OF THE INVENTION

The present invention relates to breathing abnormalities and detection thereof. In particular, a method and apparatus are described for acquiring sounds related to breathing and for identifying breathing abnormalities based on the acquired sounds.

### BACKGROUND OF THE INVENTION

Acoustic signals generated by internal body organs are transmitted to the skin, causing skin vibration. The stethoscope captures body sounds by detecting skin vibration. The stethoscope is currently employed by medical professionals to aid in the diagnosis of diseases by listening to body sounds and recognizing the patterns associated with specific diseases. However, such use of the stethoscope is limited by the episodic nature of data acquisition, as well as the limits of human acoustic sensitivity and pattern recognition. The electronic stethoscope was developed to digitally amplify the acoustic signal and aid in pattern recognition, but data acquisition is still limited by its episodic nature. Due to the weight of the stethoscope, and the lack of adequate, wearable design, the electronic stethoscope is not suitable for continuous monitoring for an active user.

The advance of computer processing led to research on computerized analysis of body sounds to identify disease states. These research studies are conducted in a controlled setting, where sensors are used to capture body sounds for computerized analysis.

Yet, to date, there are no systems available to monitor body sounds in an ambulatory, uncontrolled setting because of a multitude of design obstacles.

### SUMMARY OF THE INVENTION

An apparatus and method are for evaluating respiration. A microphone is placed in contact with a patient's skin and audio is acquired through the microphone. The acquired audio is sampled, processed and stored. At least one sound associated with respiration is identified. Abnormal respiration is identified based on frequency or duration of at least the identified sound.

### BRIEF DESCRIPTION OF THE DRAWINGS

FIG. 1A is an exploded view of a wearable device in accordance with a first exemplary embodiment of the present invention.

FIG. 1B is an exploded view of the diaphragm, diaphragm seal, and bottom housing/chestpiece assembly in accordance with a first exemplary embodiment of the present invention.

FIG. 2A-2H are perspective views that illustrate various components of the wearable device illustrated in FIG. 1.

FIG. 3 is aside view of the electronic components illustrated in FIG. 2C in accordance with an exemplary embodiment of the present invention.

FIG. 4 is a block diagram of a body sound acquisition circuit in accordance with an exemplary embodiment of the present invention.

FIG. 5 is a block diagram of sensors in accordance with an exemplary embodiment of the present invention.

FIG. 6 is a block diagram of a data processing unit in accordance with an exemplary embodiment of the present invention.

FIG. 7 is a flow chart diagram that illustrates steps that may be performed in accordance with an exemplary embodiment of the present invention.

FIG. 8 is a flow chart diagram that illustrates data processing to determine if an abnormal respiratory sound has been captured.

### DETAILED DESCRIPTION

The present invention is designed for the continuous acquisition of body sounds for computerized analysis. In contrast, existing devices for body sound acquisition are designed for episodic acquisition of body sounds for human hearing. The difference in intended use between the present invention and existing devices leads to design differences in construction materials, weight, and mechanisms of body sound acquisition. Specifically, existing designs typically require an operator to manually press the stethoscope against the skin for adequate acoustic signal acquisition. Such data acquisition is episodic, as it is limited by the duration an operator can manually press the stethoscope against the skin. In the present invention, the device is pressed against the skin using a mechanism such as adhesives or a clip to a piece of clothing worn by the patient. As such, data acquisition can occur continuously and independent of operator effort.

Existing mechanisms of body sound acquisitions include contact microphones, electromagnetic diaphragms, and air-coupler chestpieces made of metals.

Using electronic contact microphones and electromagnetic diaphragms for body sound acquisition is desirably accomplished via require tight contact between the device and the skin. Minimal movements between the device and the skin can distort the signal significantly. Thus the use of adhesive and a clip as attachment mechanisms may be precluded, as these attachment mechanisms do not offer sufficient skin contact for these types of body sound acquisition mechanisms.

The use of electromagnetic diaphragms requires more battery power in the case of continuous monitoring, which renders the design less desirable in wearable devices.

Body sound acquisition using air-coupler chestpiece is more forgiving with looser skin-device contact and unwanted movements. High density materials such as metals are used in its construction for better sound quality for human hearing. However, metallic chestpieces are too heavy for wearable applications. For example, the Littmann 3200 Electronic Stethoscope chestpiece weighs 98 grams, while an exemplary embodiment of the present invention weighs 25 grams because lightweight, lower density polymeric materials, such as acrylonitrile butadiene styrene (ABS), are used. Metals that are commonly used in chestpieces include aluminum alloy in low-cost stethoscopes and steel in premium stethoscopes. Aluminum alloys have a density of approximately 2.7 gram/cm̂3, while steels have a density of approximately 7.8 gram/cm̂3. In contrast, ABS have a density of approximately 1 gram/cm̂3. The use of lightweight, lower density air-coupler chestpiece render sound quality relatively poor for human hearing, but more than sufficient for computerized analysis.

Additionally, an exemplary embodiment of the present invention incorporates motion sensors that acquire additional physiological data used to optimize computerized body sound analysis. The physiological data include but are not limited to the phases of respiration, i.e., inhalation and exhalation, heart rate, and the degree of chestwall expansion.

A method and apparatus enable respiration of a patient to be evaluated. In accordance with an exemplary embodiment of the present invention, evaluation of patient may lead, for example, to detection of medical issues associated with respiration of a patient. The evaluation may also lead to detection of worsening lung function in patients. Exemplary patients include asthmatics and patients with chronic obstructive pulmonary disease (COPD).

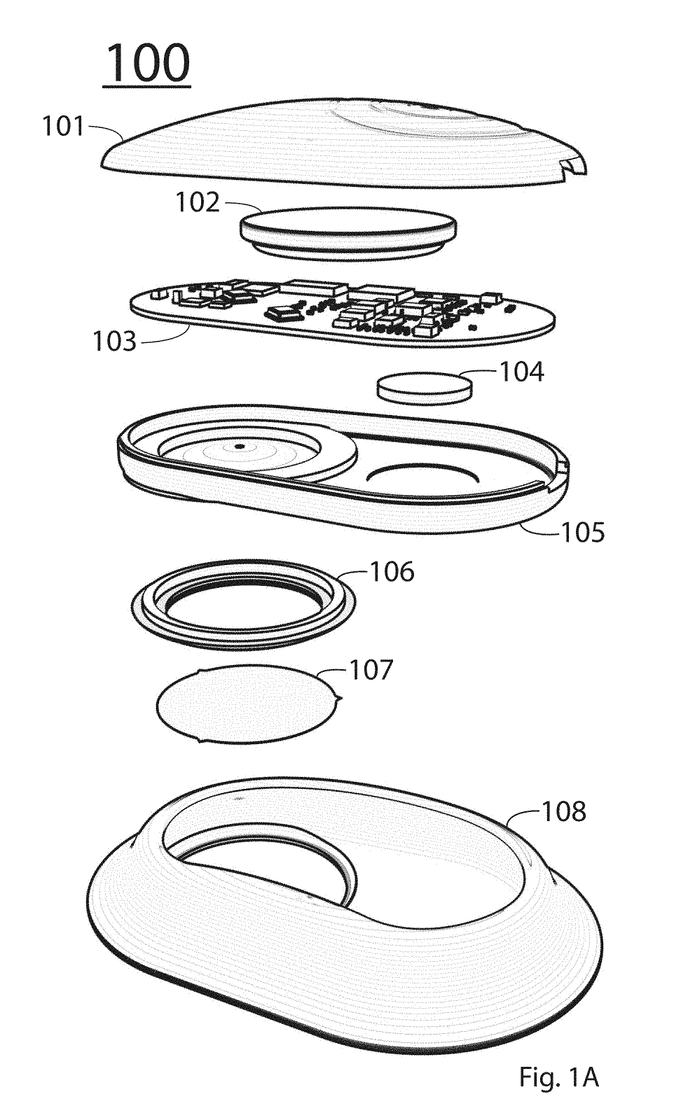

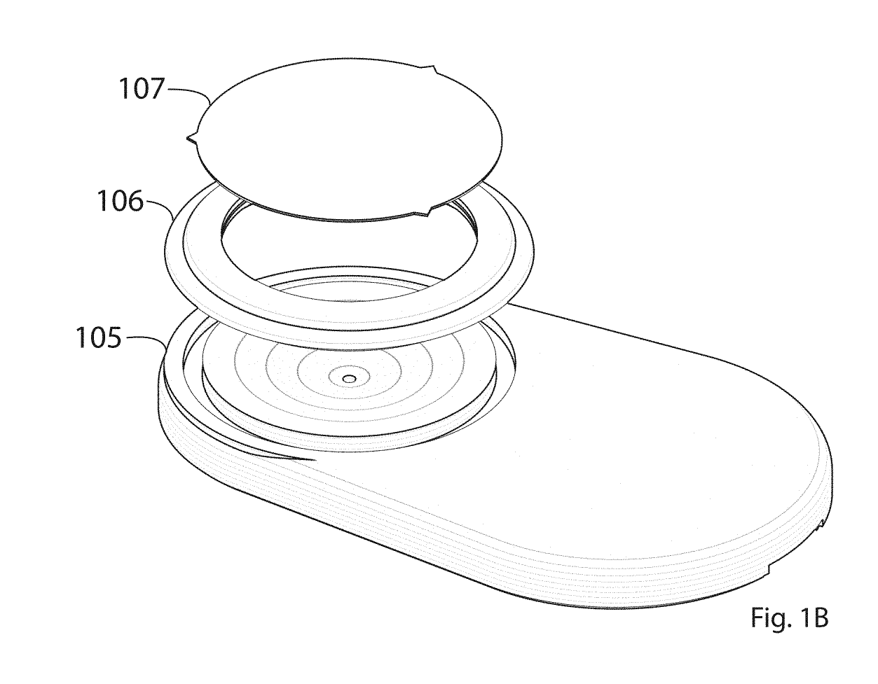

According to one aspect of the invention, a wearable device is placed in contact with a patient's body in order to receive and process sound emanating from inside the patient's body. An exploded view of an exemplary wearable device 100 is illustrated in FIG. 1A in an exploded view. Diaphragm 107 is placed in contact with a patient's skin. Diaphragm seal 106 secures diaphragm in place. Chestpiece and bottom housing 105 is placed above diaphragm 107. Electronic components 103 is placed above chestpiece 105. Top housing 101 is placed above the electronic components 103. Soft Enclosure 108 is placed below chestpiece and bottom housing 105. Several of these components are also shown in FIG. 1B. Each component of wearable device 100 will be discussed in turn.

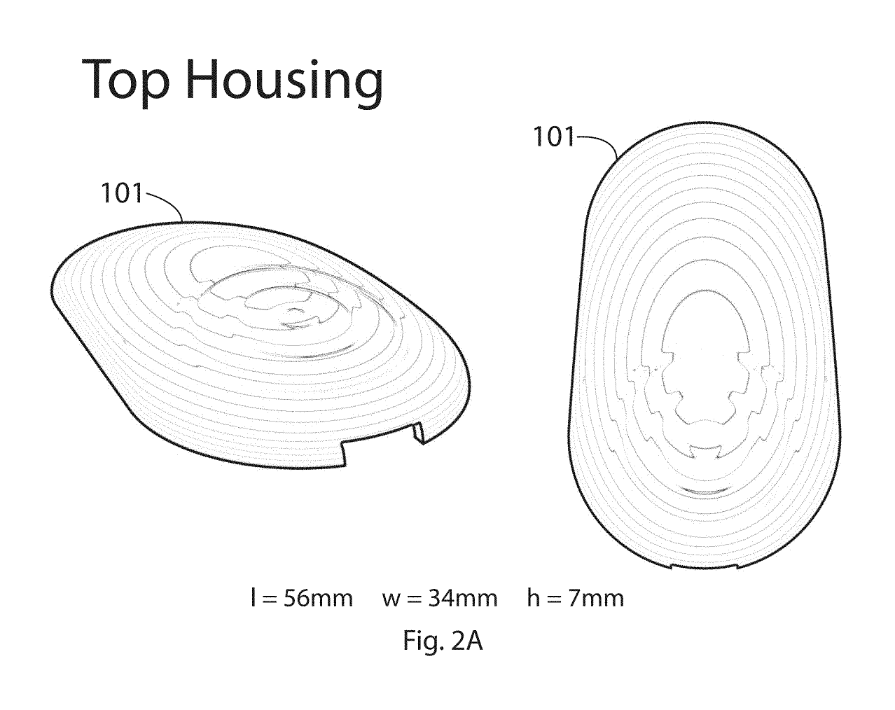

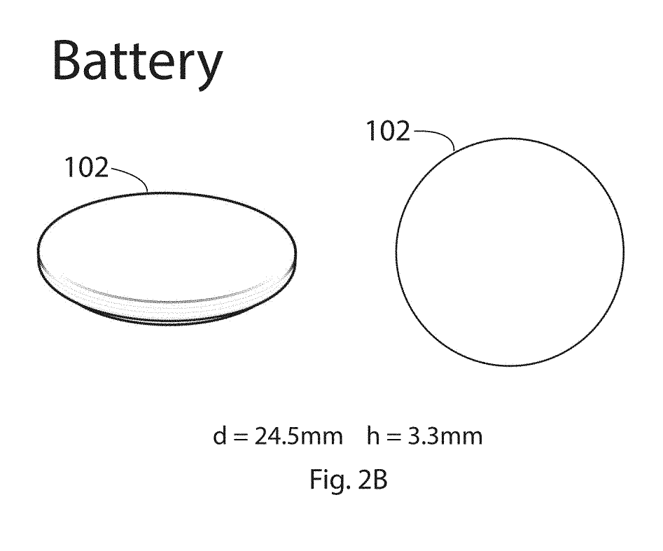

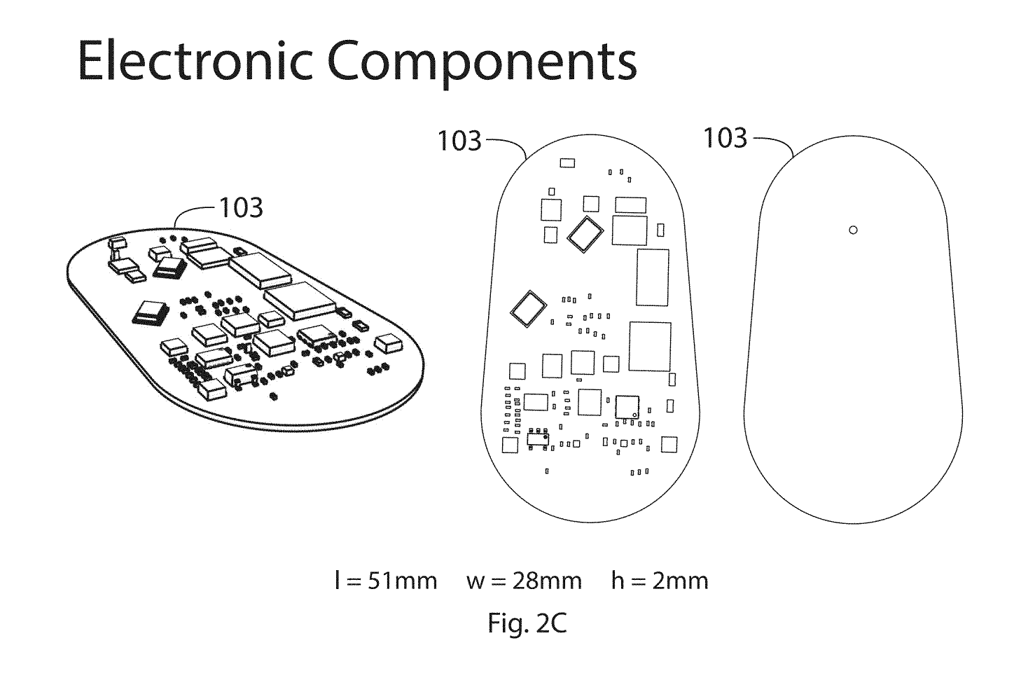

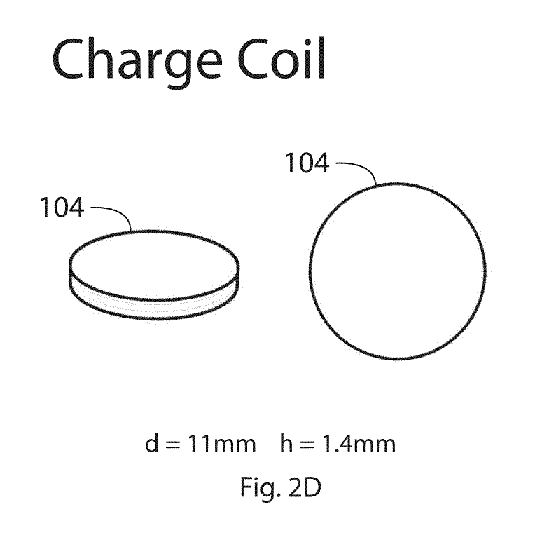

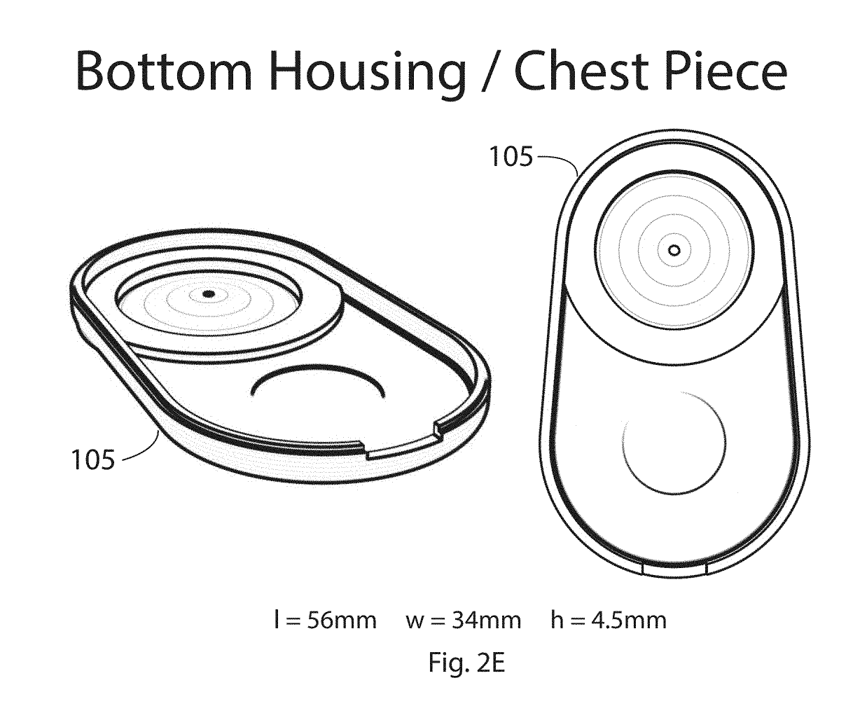

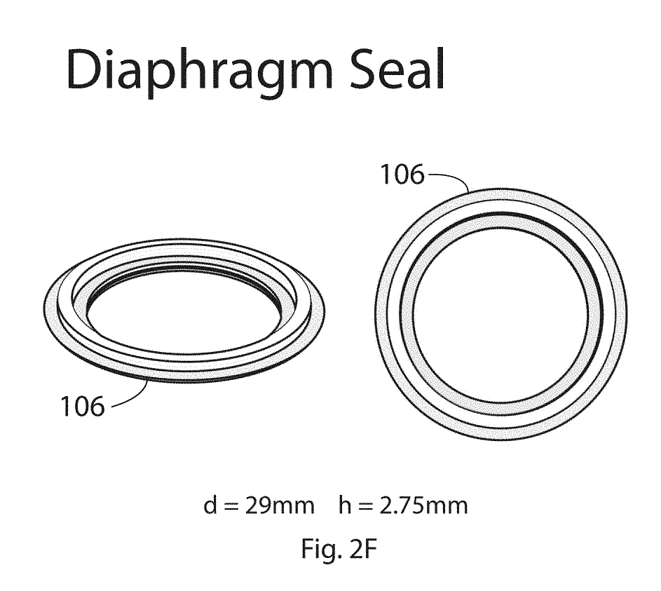

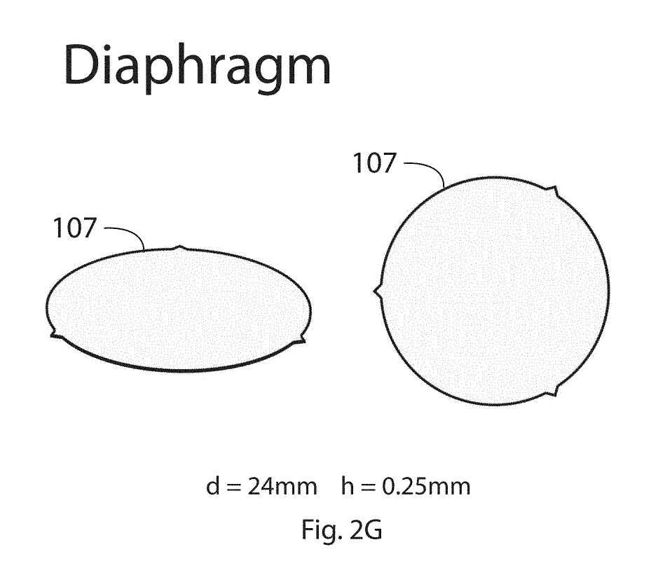

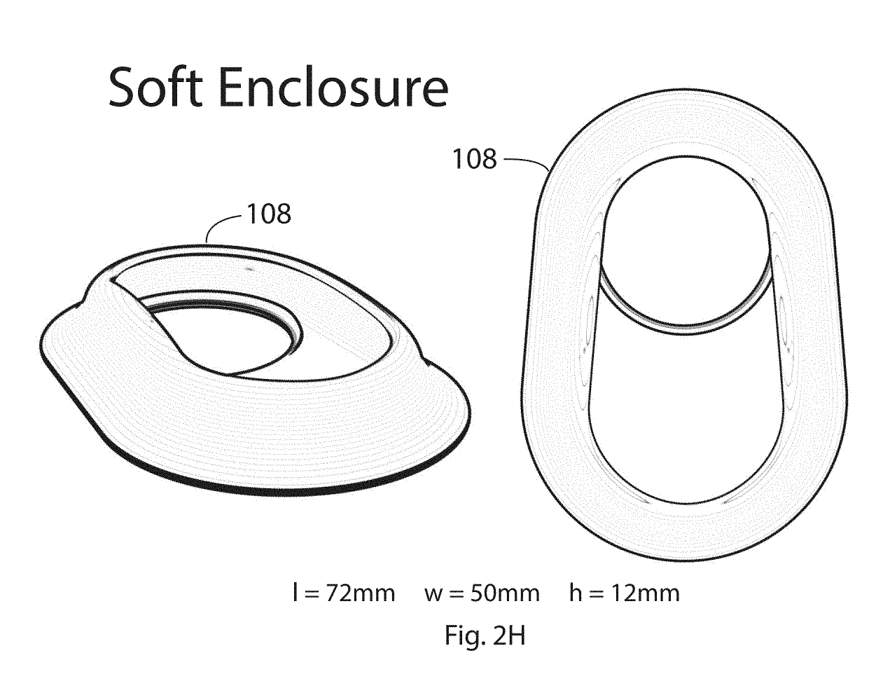

FIG. 2A illustrates exemplary top housing 101. Top housing 101 is desirably comprised of rigid, lightweight polymeric material, although other materials may be used. An exemplary size for top housing 101 is 56 mm in length, 34 mm in width, and 7 mm in height.

FIG. 2B illustrates exemplary battery 102. Battery 102 includes exemplary dimensions of 24.5 mm in diameter and 3.3 mm in height.

FIG. 2C illustrates exemplary electronic components 103 that includes exemplary dimensions of 51 mm in length, 28 mm in width, and 2 mm in height. Electronic components 130 receives audible sounds from a patient and generates data that may be used to diagnose respiratory issues. Exemplary structure and method of operation of electronic components 103 is described in detail below.

FIG. 2D illustrates exemplary charge coil 104 that includes exemplary dimensions of 11 mm in diameter and 1.4 mm in height. Charge coil 104 enables wireless charging.

FIG. 2E illustrates exemplary bottom housing and chestpiece 105 that includes exemplary dimensions of 56 mm in length, 34 mm in width, and 4.5 mm in height. Bottom housing and chestpiece 105 is desirably comprised of rigid, lightweight polymeric material, although other materials may be used. Bottom housing and chestpiece 105 is desirably comprised of one type of material although it may be melded into one piece from several types of materials.

FIG. 2F illustrates exemplary diaphragm seal 106 that includes exemplary dimensions of 29 mm in diameter and 2.75 mm in height. Diaphragm seal 106 secures diaphragm 107 to the bottom housing and chestpiece 105.

FIG. 2G illustrates exemplary diaphragm 107 that includes exemplary dimensions of 24 mm in diameter and 0.25 mm in height. Diaphragm 107 is desirably comprised of rigid, lightweight polymeric material, although other materials may be used.

FIG. 2H illustrates exemplary soft enclosure 108. Soft enclosure 108 is desirably comprised of soft silicone and includes a bottom edge designed to hold it in place. Exemplary dimensions include a length of 72 mm, a width of 50 mm, and a height of 12 mm. Soft enclosure 108 may be designed to be affixed to a patient's skin using adhesive, although other mounting mechanisms (i.e. straps or clips) may also be used.

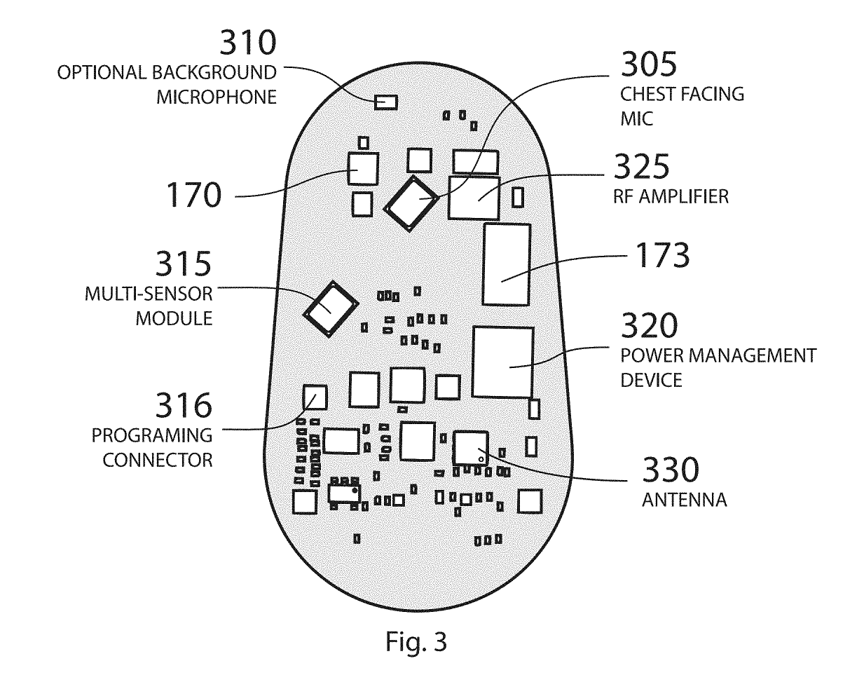

FIG. 3 provides further details regarding electronic components 103. Electronic components 103 includes Chest facing microphone 305 and optional background microphone 310 can be mounted on either side of electronic components 103. The microphone port hole of chest facing microphone 305 faces bottom housing and chestpiece 105. The microphone port hole of optional background microphone 310 faces top housing 101. Other parts included with electronic components 103 may be mounted on either side of electronic components 103 depending upon space availability. Battery 102 is included in order to power electronic components 130. Battery 102 may be a disc battery, for example, in order to provide electronic components 130 with a desirable outer thickness. Processor 170 is able to perform various operations as described below. Multi-sensor module 315 includes optional sensors including but not limited to motion sensors, thermometer, and pressure sensors. Power management device 320 optionally controls power levels within electronic components 130 in order to conserve power. RF amplifier 325 and antenna 330 optionally enable electronic components 130 to communicate with an external computing device wirelessly. Optional USB and programming connectors 316 enable wired communication with electronic components 130.

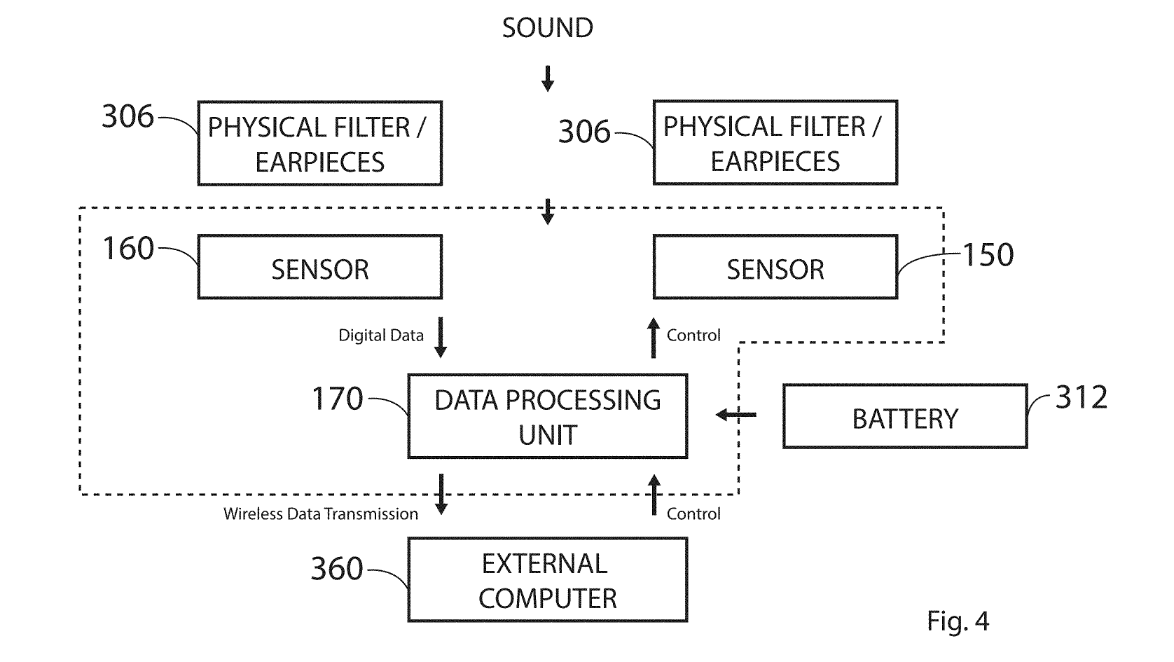

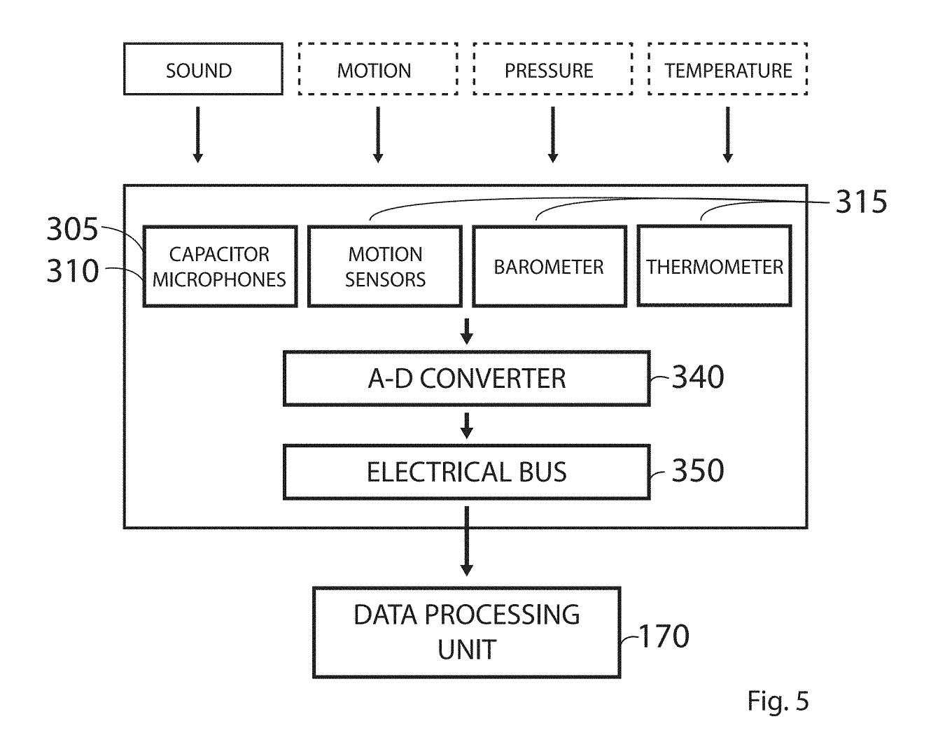

FIG. 4 is a block diagram that illustrates data acquisition circuit 150. Data acquisition circuit 150 includes sensor 160 and data processing unit 170. Received sound is received by sensor 160, which is more clearly illustrated in FIG. 5. Sensor 160 includes one or more capacitor microphones (for example) as chest facing microphone 305 and optional background microphone 310 in order to convert acoustical energy into electrical energy. Optional motion data, pressure data, and temperature data is also received by sensor 160, which is more clearly illustrated in FIG. 5. Sensor 160 includes optional multi-sensor module in order to convert analog motion, temperature, and pressure data into electrical energy. Signals from each microphone and optional multi-sensor module are transmitted to A-D converter 340 and electrical bus interface 350. Further processing is accomplished by external computer 360.

Optional physical filter(s) 306 may also be included. Exemplary filters include linear continuous-time filters, among others. Exemplary filter types include low-pass, high-pass, among others. Exemplary technologies include electronic, digital, mechanical, among others. Optional filter(s) 306 may receive sound prior to digitization, after digitization, or both.

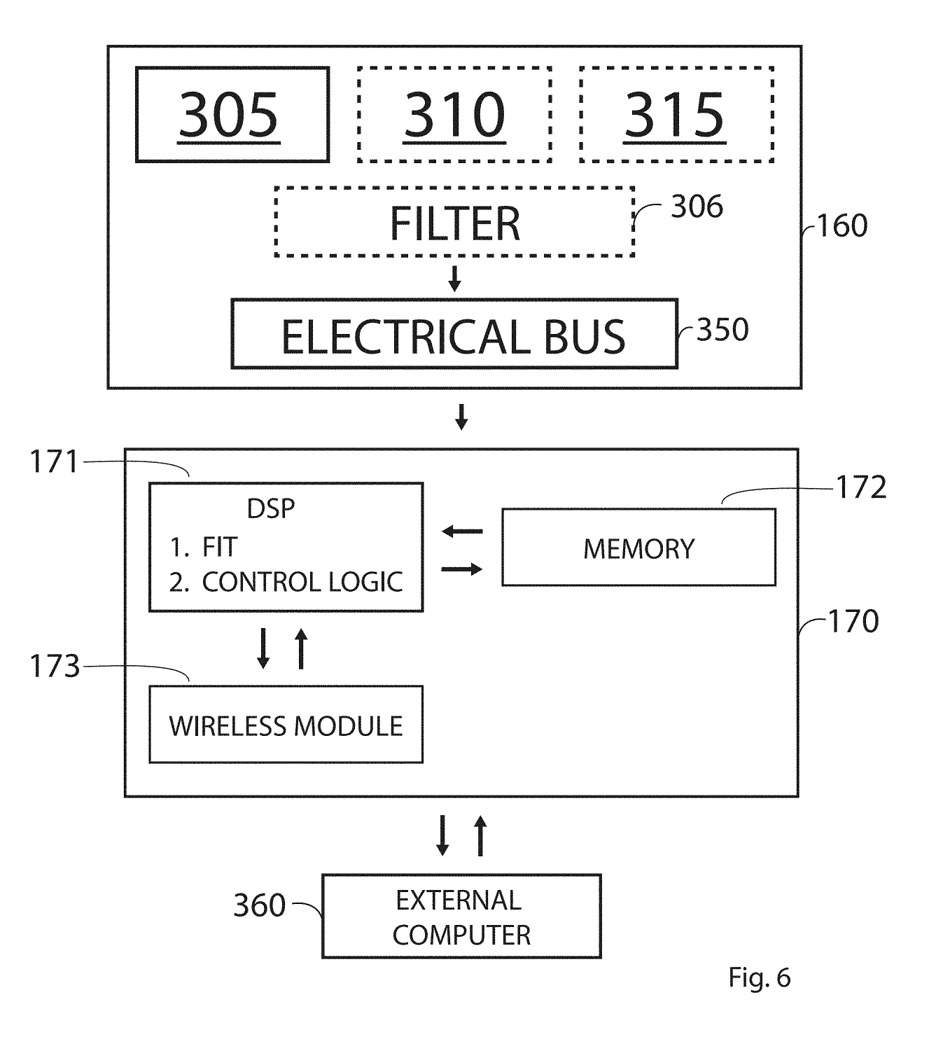

The output of electrical bus interface 350 is transmitted to data processing unit 170, which is more clearly shown in FIG. 6. Data processing unit 170 includes digital signal processor 171, memory 172 and wireless module 173 (that includes an RF amplifier and an antenna as shown in FIG. 3). Digital signal processor 171 can be programmable after manufacturing. Exemplary processors include Cypress programmable system-on-chip, field programmable gate array with integrated features, and wireless-enabled microcontroller coupled with a field programmable gate array. Wireless module 173 may use Bluetooth Low Energy as a wireless transmission standard. Wireless module 173 desirably includes an integrated balun and a fully certified Bluetooth stack. Processor 171, memory 172 and wireless module 173 are desirably integrated.

In one exemplary embodiment of the present invention, data is transferred from memory 173 to external computer 360. This is further described below.

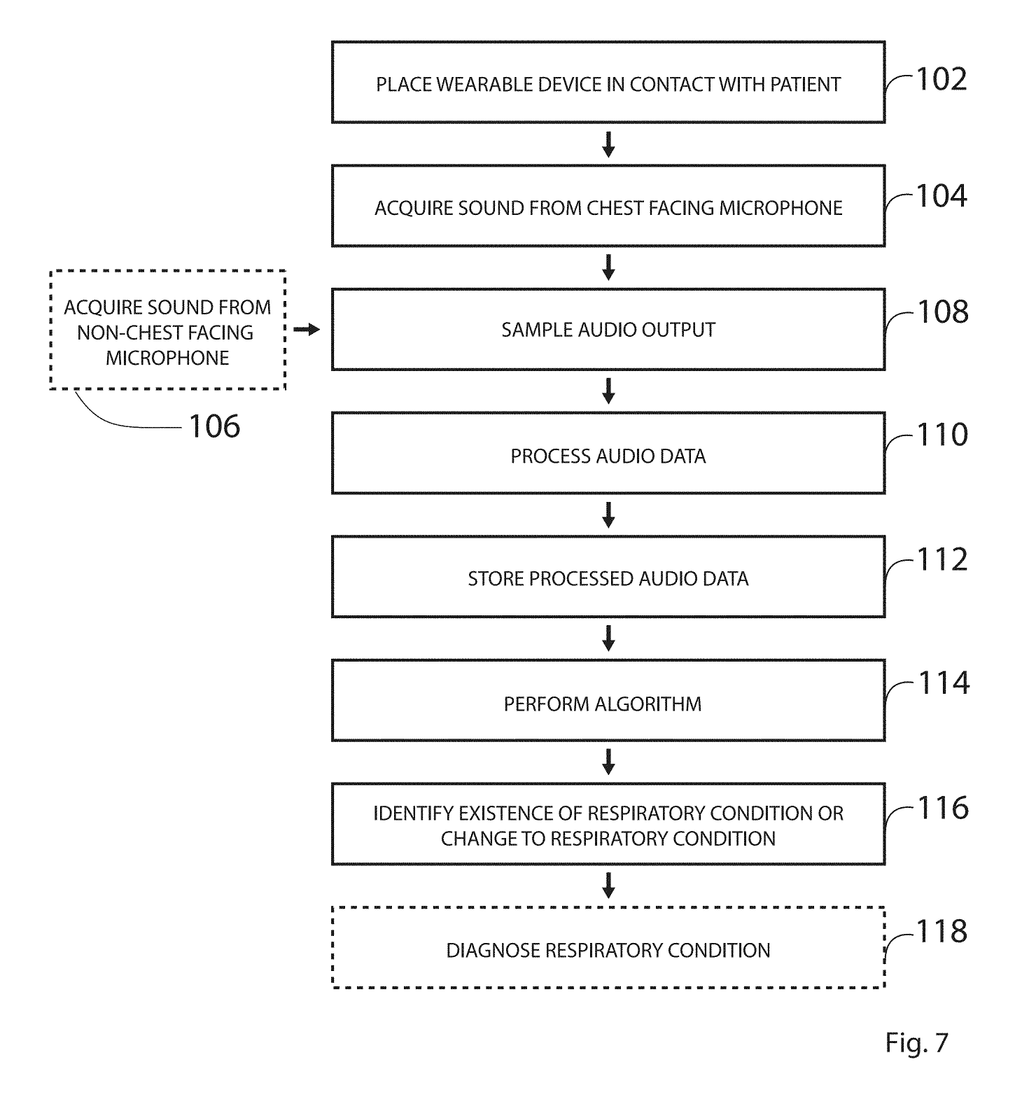

Operation of an exemplary embodiment of the present invention is illustrated by FIG. 7. At step 102, wearable device 102 is placed in contact with a patient (preferably the patient's skin). Wearable device 102 may include an adhesive to hold it in contact with the patient, although other forms of adherence may be used. Wearable device 102 is placed so that chest facing microphone 305 faces the patient and optional background microphone 310 does not face towards the patient.

At step 104, sound from chest facing microphone 305 is acquired. At optional step 106, sound from background microphone 310 is acquired. The sound optionally passes through filter 306 before being converted into electrical energy by microphone 305. After being converted to electrical energy, the sound passes through A-D converter 340 and electrical bus interface 350 before being received by digital signal processor 171. Processor 171 samples audio desirably at a minimum of 20 kHz. Sampling may occur, for example, for twenty seconds. Step 108 optionally includes the step of using the audio signals received at step 106 via microphone 310 in order to perform noise cancellation. Noise cancellation is performed using algorithms that are well known to one of ordinary skill in the art of noise cancellation.

Sampled audio data is processed at step 110. Audio data is processed in order to detect certain sounds associated with breathing (and/or associated with breathing difficulties). Processing at step 110 may include, for example, Fast Fourier Transform. Processing may also include, for example, digital low pass and/or high pass Butterworth and/or Chebyshev filters.

At optional step 112, data is stored in memory 172. FIG. 7 shows step 112 performed after step 110, but it is understood in certain circumstances that step 112 is performed concurrently with step 110 or prior to step 110. There are two types of data that are stored in memory 172. The first type of data is the “raw” data, i.e. a recording of sounds that have been sampled by microphone 305 (and that has been subjected to noise cancellation if noise cancellation is available and desired). In one exemplary embodiment of the present invention, the most recent 20 minutes of “raw” audio data is stored in memory. The data is stored in a first in, first out configuration, i.e. the oldest data is continuously deleted to make room in memory for data that is newly and continuously acquired. The second type of data that is stored in memory is processed data, i.e. data that has been subjected to a form of processing (such as time-frequency analysis) by processor 171. Examples of this type of processed data includes the examples set forth above such as Fast Fourier Transform, digital low pass and/or high pass Butterworth and/or Chebyshev filters, etc. In an exemplary embodiment of the present invention, 20 seconds of processed audio data is stored in memory 172. This data is also stored in a first in, first out configuration.

At step 114, the processed data is evaluated by processor 171 to determine if an “abnormal” respiratory sound has been captured by microphone 305. Examples of an “abnormal” respiratory sound include a wheeze, a cough, labored breathing, or some other type of respiratory sound that is indicative of a respiratory problem. Evaluation occurs as follows. In one exemplary embodiment of the present invention, the processed data (i.e. from a transform such as a Fourier transform or a wavelet transform) results in a spectrogram. The spectrogram may correspond, for example, to the 20 seconds worth of processed data that has been stored in memory 172. The spectrogram is then evaluated using a set of “predefined mathematical features”.

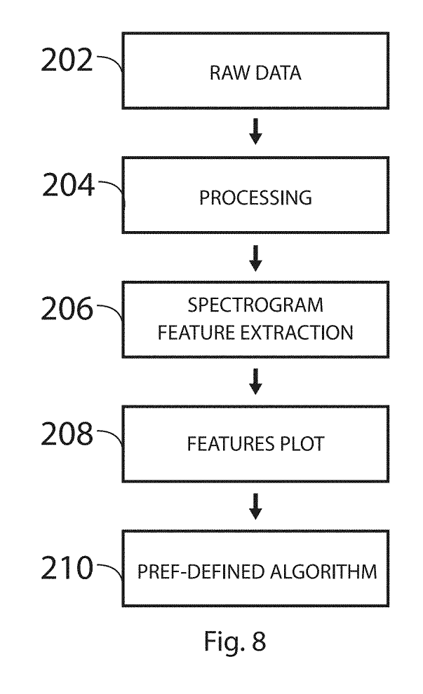

The “predefined mathematical features” are generated from multiple “predefined spectrograms”. Each “predefined spectrogram” is generated by processing data that is known to correspond to an irregular respiratory sound (such as a wheeze). A method of generating such a predefined spectrogram is illustrated by the flowchart diagram of FIG. 8 and may be performed as follows: a) a physician listens to respiratory sounds from a person using a device such as a stethoscope; b) the respiratory sounds from the person are recorded and subjected to processing such as the processing identified above; c) a spectrogram is generated based on the processing set forth above; d) the physician notes the exact time when he/she hears a sound that the physician considers to be a wheeze, e) the portion of the spectrogram that corresponds to the exact time that the physician hears the wheeze is identified, and f) that portion of the spectrogram that has been identified is used as the “predefined spectrogram.”

Once the raw data has been acquired from the patient (step 202), and is subject to audio processing (step 204), spectrogram feature extraction (step 206) may occur.

A set of mathematical features can be extracted from each predefined spectrogram. Mathematical feature extraction is known to one of ordinary skill in the art and is described in various publications, including 1) Bahoura, M., & Pelletier, C. (2004, September). Respiratory sounds classification using cepstral analysis and Gaussian mixture models. In Engineering in Medicine and Biology Society, 2004. IEMBS'04. 26th Annual International Conference of the IEEE (Vol. 1, pp. 9-12). IEEE; 2) Bahoura, M. (2009). Pattern recognition methods applied to respiratory sounds classification into normal and wheeze classes. Computers in biology and medicine, 39(9), 824-843; 3) Palaniappan, R., & Sundaraj, K. (2013, December). Respiratory sound classification using cepstral features and support vector machine. In Intelligent Computational Systems (RAICS), 2013 IEEE Recent Advances in (pp. 132-136). IEEE; 4) Mayorga, P., Druzgalski, C., Morelos, R. L., Gonzalez, O. H., & Vidales, J. (2010, August). Acoustics based assessment of respiratory diseases using GMM classification. In Engineering in Medicine and Biology Society (EMBC), 2010 Annual International Conference of the IEEE (pp. 6312-6316). IEEE; and 5) Chien, J. C., Wu, H. D., Chong, F. C., & Li, C. I. (2007, August). Wheeze detection using cepstral analysis in gaussian mixture models. In Engineering in Medicine and Biology Society. All of the above references are hereby incorporated by reference in their entireties.

The set of mathematical features are derived from the inherent power and/or frequency of the predefined spectrogram of data clusters using mathematical methods that include but are not limited to the following: data transforms (Fourier, wavelet, discrete cosine) and logarithmic analyses. The set of mathematical features extracted from each predefined spectrogram can vary by the method with which each feature in the set is extracted. These features may include, but are not limited to, frequency, power, pitch, tone, and shape of data waveform. See Lartillot, O., & Toiviainen, P. (2007, September). A Matlab toolbox for musical feature extraction from audio. In International Conference on Digital Audio Effects (pp. 237-244). This reference is hereby incorporated by reference in its entirety.

For example, a first set of two mathematical features are extracted from a predefined spectrogram using statistical mean and mode. A second set of two mathematical features are extracted from the same predefined spectrogram using statistical mean and entropy. The set of mathematical features can also vary by the number of features in each set of mathematical features. For example, a set of twenty mathematical features are extracted from a predefined spectrogram. In another example, a set of fifty mathematical features are extracted from the same predefined spectrogram. Additionally, the mathematical features may vary by the segment lengths of the predefined spectrogram with which the mathematical features are extracted. For example, a mathematical feature extracted from one-second segments of the predefined spectrogram using a statistical method is different from a mathematical feature extracted from five-second segments of the predefined spectrogram using the same statistical method.

The set of mathematical methods used to extract the “predefined mathematical features” is the “pre-specified feature extraction”. In one exemplary embodiment of the present invention, the “pre-specified feature extraction” is developed using mel-frequency cepstral coefficients and is optimized using machine learning methods that include but are not limited to the following: support vector machines, decision trees, gaussian mixed models, recurrent neural network, semi-supervised auto encoder, restricted Boltzmann machines, convolutional neural networks, and hidden Markov chain (see above references). Each machine learning method may be used alone or in combination with other machine learning methods.

The “predefined mathematical features” is derived from multiple predefined spectrograms in the following manner. An feature extraction method, as defined above, is used to extract a set of mathematical features from each predefined spectrogram corresponding to a type of respiratory sound. Multiple features are evaluated in this manner. The features are then plotted together (step 208) from multiple respiratory sound types in order to perform cluster analysis in the nth dimension (n being the number of features extracted). For example, if three features were extracted for analysis from each data file, each data file would correspond to one point in three dimensional space, each axis representing the value of a particular feature. Thereafter, one example of algorithm generation attempts to find a hyperplane in this three dimensional space that maximally separates clusters of points representing specific sound types. For example, if data points from wheeze files cluster in one corner of this three dimensional space while those from cough files cluster in another, a plane that separates these two clusters would correspond to an algorithm that distinguishes the two and is able to classify these sound types into two groups. This analysis can be extrapolated to as many features as needed, n, thereby moving the analysis into nth dimensional space. This allows differentiation of each sound type based on its unique feature set. The algorithm that generates outputs (sets of mathematical features) that are most similar to each other is selected as the “pre-specified algorithm” as described above. For example, ten sets of twenty statistical features is extracted from ten predefined spectrograms corresponding to wheezing using different algorithms. The algorithm that extracts ten sets of features that are the most similar to each other is selected as the “pre-specified algorithm” (step 210). In an exemplary graphical representation of classification, lines represent the “pre-defined algorithm” in classifying data in multiple dimensions in accordance with an exemplary embodiment of the present invention. Next, the “average” of the sets of mathematical features extracted with the “pre-specified algorithm” is selected as the “predefined mathematical features”. Here, “average” is defined by mathematical similarity between the “predefined mathematical features” and each set of mathematical features from which the “predefined mathematical features” derives from.

Evaluation of a spectrogram with a predefined spectrogram may be on several bases. A spectrogram is processed by the “pre-specified feature extraction” method to generate a set of mathematical features. The set of mathematical features is then compared to sets of “predefined mathematical features”, of which each set corresponds to a specific type of sound. If the similarity between the set of mathematical features extracted from a spectrogram and the predefined mathematical features of a type of respiratory sound goes past certain thresholds, then it is determined that the corresponding type of respiratory sound has been emitted. By saying ‘goes past” what may be meant is going above a value. What may alternatively be meant is going below a value. Thus, by portions of the spectrogram going above or below portions of the predefined spectrogram associated with possible abnormal respiratory sounds, it is determined that an abnormal respiratory sound may have occurred.

Once an irregular respiratory sound (such as a wheeze) has been identified using the “predefined mathematical features” the previous 20 (for example) minutes of accumulated raw data that has been stored in memory 172 receives “further processing.” In one exemplary embodiment of the present invention, the 20 minutes of raw data is transferred from memory 172 to external computer 360 for more robust processing. In another exemplary embodiment of the present invention, depending upon the processing power of processor 171, the 20 minutes of raw data is subjected to further processing in processor 171 without being transferred to an external computer.

The idea behind “further processing” is that a first algorithm is used to possibly identify an irregular respiratory sound and a second algorithm (more robust—i.e. that requires more significant processing than the first algorithm) is applied to the raw data to try to make a more accurate determination as to whether an irregular respiratory sound (such as a wheeze) has indeed occurred. In one exemplary embodiment of the present invention, a first algorithm generates twenty mathematical feature. A second algorithm generates fifty mathematical features and is more robust. In another exemplary embodiment of the present invention, the mathematical methods used to extract each mathematical feature in the second algorithm require more processing power than the mathematical methods used in a first algorithm. The second algorithm is more robust. In addition to using a spectrogram with the second algorithm, other factors may also be used in the analysis. Exemplary factors include: 1) user inputs, including subjective feelings, rescue inhaler use, type and frequency of medication use, current asthma status; 2) input from sensors, which include but are not limited to accelerometers, magnetometers, and gyroscopes, about a patient's current physiological status; 3) environmental inputs available from sensors, which include but are not limited to temperature sensors and barometers; and 4) environmental inputs available from an information source such as the internet. In other words, other variables are integrated into the analysis, in place of or in addition to the variables that form the basis of the analysis of the initial processed data (the 20 seconds of data, for example, discussed above).

Further processing may be performed in processor 171, external computer 360, or both, depending upon respective processing power, ability to communicate wirelessly, etc.

Thus, the further processing may include determining whether processed data has passed (i.e. above or below) boundary conditions. The boundary conditions may include one or more of any of the inputs and/or characteristics identified above. This is accomplished by pre-specified algorithms previously developed using a machine-learning approach using a deep-learning framework. This involves a multi-layer classification scheme. The variables used in the pre-specified algorithms in the external computer include, but are not limited to, the exemplary variables described above.

The “raw” data that may be stored, for example, in memory 172 provides multiple functions. For example, it provides an extended period of time for respiratory sound classification. The data may be processed into a spectrogram, and then a second algorithm may be used to analyze the spectrogram, in conjunction with other variables mentioned above. As a further example, the raw data may be used to improve the algorithm. For example, should an abnormal lung sound be recognized, it can serve as a control, and the raw data is used as a dataset to further refine (or “train”) the pre-specified algorithm.

An exemplary spectrogram based on audio data captured in accordance with an exemplary embodiment of the present invention is illustrated in FIG. 8. The top view is obtained from a microphone facing towards the patient. The bottom view is obtained from a microphone facing away from the patient.

The inventors continue to refine algorithms in accordance with exemplary embodiments of the present invention. For example, multiple sound samples are obtained and classified into different lung sounds. Next, the samples (spectrograms) are input into a pre-specified classification algorithm to generate a set of mathematical features. The difference between the output of this classification algorithm and the pre-defined mathematical features is used to refine the algorithms. The goal is ensure the classification algorithm have the variables needed to filter out unwanted noises during feature extraction. Note, the above description is based on well-described machine learning approach.

Next, the classification algorithm can be applied to additional samples containing both an audio spectrogram and additional user data defined as “boundary conditions” above. The machine learning approach in this case need not focus on feature extraction. Rather, this machine learning approach employs predictive statistical analysis. The basic concept remains the same: Difference between the classification algorithm and the pre-defined answer is used to create and adjust the weight of variables. The goal is to make a classification algorithm generalizable across different boundary conditions.

An algorithm in accordance with an exemplary embodiment of the present invention may be based on specific approaches used to train the algorithm, and the algorithm itself.

To further clarify, in one exemplary embodiment of the present invention, a respiratory condition is detected by identifying how many times a certain type of respiratory sound occurs during a time period (“frequency”). If the number of times the sound is identified in a time period goes past a threshold, then a signal is generated to indicate that an adverse respiratory condition has been detected (or that an adverse respiratory condition has gotten better or worse). By saying “goes past a threshold” what is included is meeting the threshold, going above the threshold, or going below the threshold, depending upon what adverse respiratory conditions are desired to be detected. In a further exemplary embodiment of the present invention, the number of times a certain type of respiratory sound occurs in a first time period is compared with the number of times the certain type of respiratory occurs in a second type period (the first and second time periods may or may not be overlapping, the first and second time periods may or may not be equal). For example, the number of respiratory sounds in a first time period may be compared with the number of respiratory sounds in a second time period greater than the first time period. Comparisons may be with regard to frequency, power, location in the time frame being evaluated, and/or other criteria. In one exemplary embodiment of the present invention, the first time period may be three hours and the second time period may be 18 hours. These time periods are merely exemplary.

In another exemplary embodiment of the present invention, respiratory issues are identified based on frequency of audio signal (wheeze frequency ˜300-400 Hz) and the number of times an event occurs (frequency of the event itself). When referring to threshold, we are referring to the number of times an event is detected (decompensation).

In a further exemplary embodiment of the present invention, the external computer (i.e. smartphone) modulates the frequency with which sensor 160 capture data.

The results of step 118 can be displayed and/or arranged in numerous manners. For example, it is possible to perform classification of audio data with boundaries set by user input. The classification can also be performed based on sensor data (i.e. gyroscope) included in a smartphone.

In one exemplary embodiment of the present invention, a patient is able to provide feedback—i.e. a self-assessment of the diagnosis, in order to improve accuracy of diagnosis. Regardless, historical data can be accumulated over periods of time (days, months, years) to further refine boundary conditions and models used to identify respiratory problems.

In one exemplary embodiment of the present invention, a computing device other than a smartphone may be used. Exemplary computing devices include computers, tablets, etc.

In one exemplary embodiment of the present invention, results of identification of respiratory illness, and/or changes in respiratory conditions, are provided to a patient provider. The identification and/or changes may be displayed using a variety of different user interfaces.

In one exemplary embodiment of the present invention, wearable device 100 provides an indication of remaining battery life.

In one exemplary embodiment of the present invention, near-field communication (NFC) enabled tags are used to track medication and inhaler use. A NFC enabled tag is attached to an inhaler or a medication container. After each use of the inhaler or each dose of medication, a user taps a NFC enabled computing device to the NFC enabled tag. The NFC-enabled computing device then records the time at which the tap occurs, which corresponds to the timing of the use of an inhaler or administering of a medication. The NFC-enabled computing device may include but not limited to the following: mobile phone, tablet, or as part of the electronic components 130. The output of medication-use tracking is a “boundary condition” described above.

In one exemplary embodiment of the present invention, results of identification and/or changes are pushed to a patient or to a patient provider. In another exemplary embodiment, results of identification and/or changes are pulled to a patient or to a patient provider (i.e. provided on demand).

In one exemplary embodiment of the present invention, results of identification and/or changes are provided to a patient and/or patient provider in the form of emails and/or text messages and/or other forms of electronic communication.

The sampling frequency and sampling duration set forth above are merely exemplary. In one exemplary form of the present invention, sampling frequency and/or duration may be changed.

In one exemplary embodiment of the present invention, the invention is used in combination with location technology such as GPS in order to locate location of a patient.
100 wearable device
101 top housing
102 battery
103 electronic components
104 charge coil
105 bottom housing and chestpiece
106 diaphragm seal
107 diaphragm
108 soft enclosure
150 data acquisition circuit
160 sensor
170 data processing unit
171 digital signal processor
172 memory
173 wireless module
305 chest facing microphone
306 filter
310 background microphone
312 battery
315 multi-sensor module
320 power management device
325 RF amplifier
330 antenna
340 A-D converter
350 Electrical Bus interface
360 External Computer

1. A method of evaluating respiration, said method comprising the steps of:
placing a microphone in contact with skin;
acquiring audio with the microphone;
sampling the audio acquired by the microphone;
processing the sampled audio;
storing the processed audio data;
identifying at least one type of sound associated with respiration from the processed audio data; and
identifying abnormal respiration based on at least one of frequency and duration of said sound.

2. A method according to claim 1, further comprising the step of acquiring audio from a second microphone not in contact with skin, and performing noise cancellation during the processing of the sampled audio based on audio acquired from said microphone and audio acquired from said second microphone.

3. A method according to claim 1, further comprising the step of comparing frequency of said sound within a time period to a threshold, wherein if said frequency is past said threshold than an alert relating to respiration abnormality is generated.

4. A method according to claim 3, wherein said threshold is varied.

5. A method according to claim 1, wherein said identifying is performed on a continuous basis.

6. A method according to claim 1, wherein said identifying is performed responsive to signaling a request to perform said identifying.

7. A method according to claim 1, wherein said identifying is based on identification of sounds selected from a group consisting of cough, wheeze, inhalation and exhalation.

8. A method according to claim 3, wherein said threshold is changed based on use of an asthma inhaler.

9. A method according to claim 3, wherein said threshold is changed based on use of medications

10. A method according to claim 1, further comprising the step of identifying use of an asthma inhaler based on at least one of frequency and duration of said sound.

11. A method according to claim 1, further comprising the step of displaying results of said identifying with a spectrogram.

12. A method according to claim 1, wherein said abnormal respiration is identified using pattern recognition.

13. A method according to claim 1, further comprising the step of diagnosing said abnormal respiration after identifying said abnormal respiration.

14. Apparatus for evaluating respiration, comprising:
a diaphragm,
a microphone that receives audio through said diaphragm,
an audio sampler that samples said audio;
a processor for:
identifying sound associated with respiration from said sampled audio;
identifying respiratory abnormality based on at least one of duration and frequency of said sound.

15. Apparatus according to claim 13, furthermore comprising a second microphone, wherein said processor performs noise cancellation based on audio from said microphone and audio from said second microphone.

16. Apparatus according to claim 13, wherein the processor compares frequency of said sound within a time period to a threshold, wherein if said frequency is past said threshold than an alert relating to respiration abnormality is generated.

17. Apparatus according to claim 15, wherein said threshold is varied.

18. Apparatus according to claim 13, wherein said identifying is performed on a continuous basis.

19. Apparatus according to claim 13, wherein said identifying is performed responsive to signaling a request to perform said identifying.

20. Apparatus according to claim 13, wherein said identifying is based on identification of sounds selected from a group consisting of cough, wheeze, inhalation exhalation, and loudness of respiratory sounds

21. Apparatus according to claim 15, wherein said threshold is changed based on use of an asthma inhaler.

22. Apparatus according to claim 13, wherein said processor identifies use of an asthma inhaler based on at least one of frequency and duration of said sound.

23. Apparatus according to claim 13, wherein said processor displays results of said identifying with a spectrogram.

24. Apparatus according to claim 13, wherein said abnormal respiration is identified using pattern recognition.

25. Apparatus according to claim 13, wherein said processor diagnoses said abnormal respiration after identifying said abnormal respiration.
